(19) United States
(12) Patent Application Publication
(10) Pub. No.: US 2004/0143333 A1
(43) Pub. Date: Jul. 22, 2004

(54) Prosthetic spinal disc nucleus with elevated swelling rate

(76) Inventors: Allison C. Bain, Whitehouse Station, NJ (US); Tara Nicole Sherman, Cottage Grove, MN (US); Britt Keenan Norton, Eden Prairie, MN (US)

Correspondence Address: Scott D. Rothenberger, DORSEY & WHITNEY LLP, Intellectual Property Department, 50 South Sixth Street, Suite 1500, Minneapolis, MN, 55402-1498, US

(21) Appl. No.: 10/723,718

(22) Filed: Nov. 25, 2003

Related U.S. Application Data
(60) Provisional application No. 60/429,333, filed on Nov. 26, 2002.

(51) Int. Cl. A61F 2/44

(52) U.S. Cl. 623/17.16; 264/343

(57) ABSTRACT

A method of manufacturing a prosthetic spinal disc nucleus. The including forming a hydrogel core from a hydrogel material in a natural state. The hydrogel material in the natural state is characterized by a natural swelling rate. The hydrogel is treated in an alkaline solution having a pH of at least about 8. This treatment transitions the hydrogel core from the natural state to a treated state characterized by an elevated swelling rate. The elevated swelling rate is greater that the natural swelling rate. The resultant, treated hydrogel core forms at least a portion of a prosthetic spinal disc nucleus that is otherwise sized for insertion into a spinal disc nucleus cavity. In one particular embodiment, the hydrogel core is inserted into a constraining jacket. Another aspect of the present invention relates to a prosthetic spinal disc nucleus including a hydrogel core having the elevated swelling rate.

### CROSS-REFERENCE TO RELATED APPLICATION

The present application claims priority to U.S. provisional patent application 60/429,333 filed Nov. 26, 2002, which is incorporated herein by reference in its entirety.

### BACKGROUND OF THE INVENTION

The vertebral spine is the axis of the skeleton upon which all of the body parts “hang”. In humans, the normal spine has seven cervical, twelve thoracic and five lumbar segments. The lumbar segments sit upon a sacrum, which then attaches to a pelvis, in turn supported by hip and leg bones. The bony vertebral bodies of the spine are separated by intervertebral discs, which act as joints, but allow known degrees of flexion, extension, lateral bending and axial rotation.

The typical vertebra has a thick interior bone mass called the vertebral body, and a neural (vertebral) arch that arises from a posterior surface of the vertebral body. Each neural arch combines with the posterior surface of the vertebral body and encloses a vertebral foramen. The vertebral foramina of adjacent vertebrae are aligned to form a vertebral canal, through which the spinal sac, cord and nerve rootlets pass. The portion of the neural arch that extends posteriorly and acts to protect a posterior side of the spinal cord is known as the lamina. Projecting from the posterior region of the neural arch is a spinous process. The central portions of adjacent vertebrae are each supported by an intervertebral disc.

The intervertebral disc primarily serves as a mechanical cushion between the vertebral bones, permitting controlled motions within vertebral segments of the axial skeleton. The normal disc is a unique, mixed structure, comprised of three component tissues: the nucleus pulposus (“nucleus”), the anulus fibrosus (“anulus”), and two opposing vertebral endplates. The two vertebral endplates are each composed of thin cartilage overlying a thin layer of hard, cortical bone that attaches to the spongy, richly vascular, cancellous bone of the vertebral body. The endplates thus serve to attach adjacent vertebrae to the disc. In other words, a transitional zone is created by the endplates between the malleable disc and the bony vertebrae.

The anulus of the disc is a tough, outer fibrous ring that binds together adjacent vertebrae. This fibrous portion, which is much like a laminated automobile tire, is generally about 10 to 15 millimeters in height and about 15 to 20 millimeters in thickness. The fibers of the annulus consist of 15 to 20 overlapping multiple plies, and are inserted into the superior and inferior vertebral bodies at roughly a 30-degree angle in both directions. This configuration particularly resists torsion, as about half of the angulated fibers will tighten when the vertebrae rotate in either direction, relative to each other. The laminated plies are less firmly attached to each other.

Immersed within the anulus, positioned much like the liquid core of a golf ball, is the nucleus. The anulus and opposing endplates maintain a relative position of the nucleus in what can be defined as a nucleus cavity. The healthy nucleus is largely a gel-like substance having a high water content, and similar to air in a tire, serves to keep the anulus tight yet flexible. The nucleus-gel moves slightly within the anulus when force is exerted on the adjacent vertebrae with bending, lifting, etc.

The nucleus and the inner portion of the anulus have no direct blood supply. In fact, the principal nutritional source for the central disc arises from circulation within the opposing vertebral bodies. Microscopic, villous-like fingerlings of the nuclear and anular tissue penetrate the vertebral endplates and allow fluids to pass from the blood across the cell membrane of the fingerlings and then inward to the nuclear tissue. These fluids are primarily body water and the smallest molecular weight nutrients and electrolytes.

The natural physiology of the nucleus promotes these fluids being brought into, and released from, the nucleus by cyclic loading. When fluid is forced out of the nucleus, it passes again through the endplates and then back into the richly vascular vertebral bodies. The cyclic loading amounts to daily variations in applied pressure on the vertebral column (e.g., body weight and muscle pull) causing the nucleus to expel fluids, followed by periods of relaxation and rest, resulting in fluid absorption or swelling by the nucleus. Thus, the nucleus changes volume under loaded and non-loaded conditions. Further, the resulting tightening and loosening effect on the anulus stimulates the normal anulus collagen fibers to remain healthy or to regenerate when torn, a process found in all normal ligaments related to body joints. Notably, the ability of the nucleus to release and imbibe fluids allows the spine to alter its height and flexibility through periods of loading or relaxation. Normal loading cycling is thus an effective nucleus and inner anulus tissue fluid pump, not only bringing in fresh nutrients, but perhaps more importantly, removing the accumulated, potentially autotoxic by-products of metabolism.

The spinal disc may be damaged due to trauma or a disease process. A disc herniation occurs when the anulus fibers are weakened or torn and the inner tissue of the nucleus becomes permanently bulged, distended, or extruded out of its normal, internal anular confines. The mass of a herniated or “slipped” nucleus can compress a spinal nerve, resulting in leg pain, loss of muscle control, or even paralysis. Alternatively, with discal degeneration, the nucleus loses its water binding ability and deflates, as though the air had been let out of a tire. Subsequently, the height of the nucleus decreases, causing the anulus to buckle in areas where the laminated plies are loosely bonded. As these overlapping laminated plies of the anulus begin to buckle and separate, either circumferential or radial anular tears may occur, which may contribute to persistent and disabling back pain. Adjacent, ancillary spinal facet joints will also be forced into an overriding position, which may create additional back pain.

Whenever the nucleus tissue is herniated or removed by surgery, the disc space will narrow and may lose much of its normal stability. In many cases, to alleviate pain from degenerated or herniated discs, the nucleus is removed and the two adjacent vertebrae are surgically fused together. While this treatment alleviates the pain, all distal motion is lost in the fused segment. Ultimately, this procedure places greater stress on the discs adjacent the fused segment as they compensate for the lack of motion, perhaps leading to premature degeneration of those adjacent discs. A more desirable solution entails replacing in part or as a whole the damaged nucleus with a suitable prosthesis having the ability to complement the normal height and motion of the disc while stimulating the natural disc physiology.

The first prostheses embodied a wide variety of ideas, such as ball bearings, springs, metal spikes and other perceived aids. These prosthetic discs were designed to replace the entire intervertebral disc space, and were large and rigid. Beyond the questionable efficacy of those devices were the inherent difficulties encountered during implantation. Due to their size and inflexibility, these first generation devices required an anterior implantation approach as the barriers presented by the lamina and, more importantly, the spinal cord and nerve rootlets during posterior implantation, could not be avoided. Recently, smaller and more flexible hydrogel based prosthetic nucleus devices have been developed. These prosthetics are generally implanted in a dehydrated state. Upon insertion, the hydrophilic prosthesis will expand, thus providing support to the spinal cord area and also relief to the patient. However, upon implantation, the prosthesis often takes a week or longer to fully hydrate and remains an issue in terms of patient rehabilitation time and/or often requires that the patient remain stationary for extended periods of time and/or, consequently, in a hospital setting.

Therefore, a need exists for a prosthetic spinal disc nucleus implantable in a form having an enhanced swelling rate and equilibrium swelling level.

### SUMMARY OF THE INVENTION

One aspect of the present invention relates to a method of manufacturing a prosthetic spinal disc nucleus. The method includes forming a hydrogel core from a hydrogel material in a natural state. The hydrogel material in the natural state is characterized by a natural swelling rate. The hydrogel is treated in an alkaline solution, i.e., a solution having a pH of greater than about 7. This treatment transitions the hydrogel core from the natural state to a treated state characterized by an elevated swelling rate. The elevated swelling rate is greater that the natural swelling rate. The resultant, treated hydrogel core forms at least a portion of a prosthetic spinal disc nucleus that is otherwise sized for insertion into a spinal disc nucleus cavity. In one embodiment, the hydrogel core is inserted into a constraining jacket. Another aspect of the present invention relates to a prosthetic spinal disc nucleus including a hydrogel core having the elevated swelling rate.

Yet another aspect of the present invention relates to a method of manufacturing a prosthetic spinal disc nucleus. The method includes forming a hydrogel core from a hydrogel material in a natural state. The hydrogel material in the natural state is characterized by a natural equilibrium swelling level. The hydrogel is treated in a solution having a pH of at least about a pH of 7.4. This treatment transitions the hydrogel core from the natural state to a treated state characterized by an elevated equilibrium swelling level. The elevated equilibrium swelling level is greater that the natural equilibrium swelling level. The resultant, treated hydrogel core forms at least a portion of a prosthetic spinal disc nucleus that is otherwise sized for insertion into a spinal disc nucleus cavity. Yet another aspect of the present invention relates to a prosthetic spinal disc nucleus including a hydrogel core having the elevated equilibrium swelling level.

### BRIEF DESCRIPTION OF THE DRAWING

FIG. 1 is a perspective view of a prosthetic spinal disc nucleus in accordance with the present invention; and

FIGS. 2-4 are graphs illustrating elevated swelling rates and equilibrium swelling levels provided with a hydrogel core of the present invention.

### DESCRIPTION OF THE INVENTION

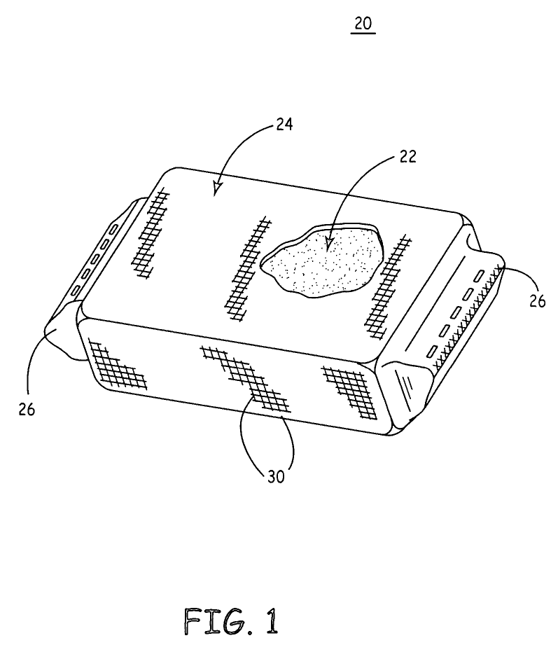

One embodiment of a spinal prosthetic disc nucleus 20 is shown in FIG. 1. As made clear below, a prosthetic disc nucleus in accordance with the present invention can assume a variety of constructions, but generally includes a hydrogel core 22 having certain characteristics. Pursuant to one embodiment, the prosthetic disc nucleus 20 further includes a constraining jacket 24 that is secured about the hydrogel core 22 by closures 26 located at opposite ends of the constraining jacket 24.

The construction of the prosthetic disc nucleus 20, including the hydrogel core 22 and the constraining jacket 24, can assume a number of different shapes and sizes. Examples of acceptable constructions are provided in Ray et at., U.S. Pat. No. 5,824,093 and U.S. patent application Ser. No. 09/090,820, the teachings of which are incorporated herein by reference. In general terms, the hydrogel core 22 is generally formulated as a hydrogel copolymer, such as an acrylamide-based copolymer and subjected to certain fabrication conditions described below.

Suitable hydrogels used in the construction of such cores 22, include, for example but are not limited, to poly(acrylamides), poly(N-vinyl-2-pyrrolidones, polyacrylates, poly(vinyl alcohols), poly(ethylene oxides).

In particular, an acrylamide/acrylonitrile block co-polymer can be used. Alternatively, the hydrogel core 22 can be any hydrophilic acrylate derivative with a unique multi-block co-polymer structure or any other hydrogel material having the ability to deform and reform in a desired fashion in response to placement and removal of loads thereon. For example, the hydrogel core 22 can be formulated as a mixture of polyvinyl alcohol and water. Much like a normal disc nucleus, the hydrogel core 22 will initially swell from the dehydrated state as it absorbs fluid. When fully hydrated, the hydrogel core 22 will have a water content of 25%-90%. The hydrogel material used for the hydrogel core 22 in a particular embodiment is manufactured under the trade name Hypan® by Hymedix International, Inc. of Dayton, N.J.

In addition to providing for varying water contents and volumes, the hydrogel core 22 material generally allows the prosthetic disc nucleus 20 to be manufactured to assume different shapes in either the dehydrated state or the final hydrated state. For example, the hydrogel core 22 can be fabricated to have an elongated, rectangular shape in the dehydrated state shown in FIG. 1. Alternatively, the hydrogel core 22 can be angled, wedged, circular, etc. Even further, the hydrogel core 22 can be formed to assume an irregular shape, such as a shape corresponding generally with a shape of a disc nucleus. Due to shape memory characteristic associated with many hydrogel materials, such as Hypan®, the hydrogel core 22 can be formed to a first shape in the final hydrated state and a second shape in the dehydrated state. For example, the hydrogel core 22 can be formed to assume a generally rectangular shape in the dehydrated state, subsequently hydrating and expanding to a tapered, wedged configuration in the final hydrated state.

Beyond the general hydrogel materials described above, the hydrogel core 22 of the present invention is characterized by an elevated swelling rate and/or elevated equilibrium swelling level, with these characteristics being imparted by subjecting/treating the hydrogel core 22 during the manufacture thereof. By way of background, polyacrylonitrile-based hydrogels, such as those typically used in hydrogel-type prosthetic disc nucleus products are fabricated by first dissolving the hydrogel raw material in an organic solvent such as dimethyl sulfoxide (DMSO). The relative amounts of hydrogel and solvent used will depend on the final mechanical properties desired. The polymer solution is then molded or cast into a desired shape and cured at an elevated temperature. Once the hydrogel core is in the final shape, the remaining organic solvent is removed via solvent exchange with water. The thus-formed core can be subjected to other processing, such as placement of loading forces that impart a desired, dehydrated shape different from the molded shape of the hydrogel core 22. However, the inherent, natural characteristics of the hydrogel material, and in particular the natural swelling rate (i.e., the rate at which the hydrogel core 22 imbibes fluids) and the natural equilibrium swelling level (i.e., the weight of the hydrogel core 22 once hydration is essentially complete) are not affected. As used throughout this specification, the term “natural swelling rate” is in reference to a swelling rate of the hydrogel core 22 following normal fabrication. The term “natural equilibrium swelling level” is in reference to an equilibrium swelling level of the hydrogel core 22 following normal fabrication. The present invention provides the hydrogel core 22 with enhanced (e.g., elevated) swelling rate and equilibrium swelling level characteristics by immersing the hydrogel core 22 in a solution having a pH between about 7.4 and about 14. For comparison, the natural equilibrium swelling level is used to calculate the percent hydration for an enhanced hydrogel sample as follows:

$$\frac{( current weight - initial weight ) / ( initial weight )}{\begin{matrix}( natural equilibrium swelling level - natural dehydrated weight ) / \\ ( natural dehydrated weight )\end{matrix}}$$

The percent increase in the swelling rate is based on the time it takes to reach the natural full hydration (based on assumption that 95% or greater hydration is considered fully hydrated).

In particular, following normal fabrication, the hydrogel core 22 or device is placed in an buffer solution (or alkaline buffer solution) having a pH of at least about 7.4. In one embodiment, the hydrogel core 22 is at least partially dehydrated, more particularly, dehydrated, prior to immersion in the buffer solution. Alternatively, the hydrogel core 22 or device can be hydrated and then treated with the buffer solution. The hydrogel core 22 is then allowed to at least partially hydrate or swell, more particularly fully hydrate or swell, in the buffer solution. Following this treatment, the swelling rate and/or the equilibrium swelling level of the “treated” hydrogel core 22 is/are elevated. Subsequently, the treated hydrogel core 22 is processed in accordance with the particular prosthetic disc nucleus 20 construction, such as, in one particular embodiment, dehydrating the treated hydrogel core 22 and placing it within the constraining jacket 24.

In one embodiment, alkaline treatment of the hydrogel core 22 occurs in a solution having a pH of at least about 7.4, more particularly of at least about 8, more particularly at least about 9, and even more particularly of at least about 10. Alternatively, the solution can have a pH of about 11 or about 14. Thus, a pH range of from between about 7.4 and about 14 can be utilized for enhanced swelling rates. For example, in one embodiment, the hydrogel core 22 is treated in a solution of NaOH having a pH of about 10. Although the alkaline solution has been described as being derived from NaOH, any suitable base and/or pH buffering system is acceptable within the general pH range of between about 7.4 and about 14 pH units.

It has surprisingly been found that by controlling the dwell time of the hydrogel core 22 in the alkaline solution, desired increases in the swelling rate and equilibrium swelling level can be achieved. More particularly, while in one embodiment, the hydrogel core 22 is allowed to fully hydrate in the alkaline solution, in other embodiments, the hydrogel core 22 is removed from the alkaline solution prior to full hydration. For example, in one embodiment, a hydrogel core having a dehydrated weight of approximately 1.5 grams is immersed (fully dehydrated) in an alkaline solution having a pH of about 10 for approximately 48 hours (as compared to approximately 120 hours required to achieve full hydration). The resulting, treated hydrogel core 22 exhibits elevated swelling rate and equilibrium swelling level characteristics (as compared to the natural swelling rate and equilibrium swelling level values), but at a lesser amount than would otherwise be achieved if allowed to imbibe to full hydration in the alkaline solution. Alternatively, other controlled dwell times can be employed. Regardless, by removing the hydrogel core 22 from the alkaline solution prior to full hydration, the resultant swell rate and equilibrium swell level can be controlled to desired values.

Regardless of the exact pH and dwell time parameters, and not to be limited by theory, it is believed that the alkaline solution treatment step described above modifies the hydrogel core 22 material via chelation with the ions present within the pH buffer. In some instances, salts of the amides, carboxyls, and/or hydroxyls can result. These so-retained salts and/or chelates do not affect the efficacy of the hydrogel core 22 within a human body, but provide the highly beneficial swelling rate and equilibrium swelling 20 level improvements desired. The ions (metallic or organic) can be removed or released from the hydrogel polymer matrix through a known extraction processes if deemed necessary.

In one embodiment, the treated hydrogel core 22 exhibits an elevated swelling rate that is at least about 10% greater, more particularly at least about 50% greater, even more particularly at least about 75% greater than the natural swelling rate. Similarly, the treated hydrogel core 22 exhibits an elevated equilibrium swelling level that is at least about 10% greater, more particularly at least about 15% greater, even more particularly at least about 25% greater than the natural equilibrium swelling level. In this regard, one acceptable manner to characterize swelling rate is to dehydrate the hydrogel core 22, place the dehydrated hydrogel core in water (so that the hydrogel core 22 hydrates) and then periodically weigh the hydrogel core 22 as it hydrates. With this in mind, and by way of example, an approximately 1.5 gram sample (dehydrated) of untreated Hypan®, available from Hymedix International Inc., of Dayton, N.J., will achieve 95% hydration after approximately 72 hours. In contrast, the hydrogel core 22 in accordance with the present invention (having a dehydrated weight of approximately 1.5 grams) will achieve 95% hydration in less than about 55 hours, more particularly less than about 35 hours, and even more particularly less than about 20 hours. Thus, the hydrogel core 22 in accordance with the present invention is characterized by an elevated swelling rate that is at least about 125% of the natural swelling rate, more particularly at least about 150% of the natural swelling rate, and even more particularly at least 175% of the natural swelling rate (Percentages based on the assumption that 95% or greater hydration is considered fully hydrated). Similarly, the untreated, approximately 1.5 gram (dehydrated) Hypan® sample has a natural equilibrium swelling level of approximately 3.0 grams. In contrast, an approximately 1.5 gram (dehydrated) version of the hydrogel core 22 in accordance with the present invention is characterized by an elevated equilibrium swelling level of at least about 3.2 grams, more particularly at least about 3.5 grams, even more particularly at least about 4.0 grams. Thus, the hydrogel core 22 in accordance with the present invention has an elevated equilibrium swelling level that is at least about 110% of the natural equilibrium swelling level, more particularly at least about 115% of the natural equilibrium swelling level, even more particularly at least about 125% of the natural equilibrium swelling level.

The treatment step described above can be performed at various points during manufacture of the prosthetic disc nucleus 20 or device. The buffer solution treatment step can occur before or after placement within the constraining jacket 24.

Again, with reference to one embodiment of the prosthetic disc nucleus 20, the constraining jacket 24 is generally a flexible tube made of tightly woven, high tenacity polymeric fabric. For example, in one embodiment, high molecular weight polyethylene is used as the weave material for the constraining jacket 24. However, polyester or any other high tenacity polymeric material can be employed, and carbon fiber yarns, ceramic fibers, metallic fibers, etc., are also acceptable.

The constraining jacket 24 can be made of fibers that have been highly oriented along their length. As a result, the constraining jacket 24 material while flexible, has little elasticity or stretch. The constraining jacket 24 defines a generally fixed maximum volume including a generally fixed length (x-axis of FIG. 1). In one embodiment, the generally fixed maximum volume of the constraining jacket 24 is less than a theoretical volume of the treated hydrogel core 22 if allowed to completely hydrate without constraint. Thus, because the treated hydrogel core 22 has a fully hydrated volume greater than that of the constraining jacket 24, the constraining jacket 24 will be tight about the hydrogel core 22 in the final hydrated state.

The woven construction of the constraining jacket 24 creates a plurality of small openings 30 (shown generally in FIG. 1). Each of the plurality of small openings 30 is large enough to allow hydration of the hydrogel core 22, but are small enough to prevent the hydrogel core 22 from escaping. Each of the plurality of small openings 30 has an average diameter of 10 micrometers, although other dimensions are acceptable. In this regard, although the constraining jacket 24 has been described as having a woven configuration, any other configuration having a semi-permeable or porous attribute can be employed. Of course it should be understood that the jacket can affect swelling percentages as it may constrain the hydrogel core. This can be controlled by the construction of the jacket and allowance can be made for the degree of swelling desired.

As described in greater detail below, following implantation, the constraining jacket 24 serves to constrain hydration and expansion of the hydrogel core in a predetermined, desired fashion. Alternatively, the prosthetic disc nucleus 20 can be configured to control, constrain and/or simply contain the hydrogel core 22 with components/structures different from the constraining jacket 24. For example, the hydrogel core 22 can be disposed within a flexible, permeable bag having a volume slightly greater than a volume of a nucleus cavity into which the prosthetic disc nucleus 20 is implanted. Even further, the hydrogel core 22 can be contained within a more rigid structure. Even further, the hydrogel core 22 can be implanted without a separate enclosure body, such that the constraining jacket 24 is eliminated.

The prosthetic spinal disc nucleus is inserted into the intradiscal cavity following partial or complete removal of the native spinal disc nucleus material, using any of the art recognized surgical approaches and instruments known for treatment of spinal disc disorders. The prosthetic spinal disc nucleus is inserted in a dehydrated or partially hydrated state, so as to minimize the size of the incision through the spinal disc annulus and to minimize overall surgical access. The present invention provides the advantage that the prosthetic spinal disc nucleus having a hydrogel core, after treatment with an alkaline solution during manufacture as described herein, exhibits an elevated swelling rate to its final swelling level as it absorbs water from the surrounding bodily fluids. Upon insertion into the intradiscal cavity via any one of art recognized surgical techniques, the prosthetic spinal disc nucleus may be optionally treated with about 10 mL of water to help effect hydration.

Thus the present invention provides the advantage that an inserted spinal disc nucleus will expand more rapidly into place in comparison to similar implants currently available. This provides the further advantages of increased patient comfort and a decrease in the amount of time the patient remains in the hospital. Additionally, the patient is able to ambulate more quickly and is not required to remain prone for extended periods of time until the implant has achieved desired swelling characteristics.

### EXAMPLES AND COMPARISONS

### Example 1

Pre-Swelling Hydrogel Pellets in pH 10 and 12 Buffer Solutions

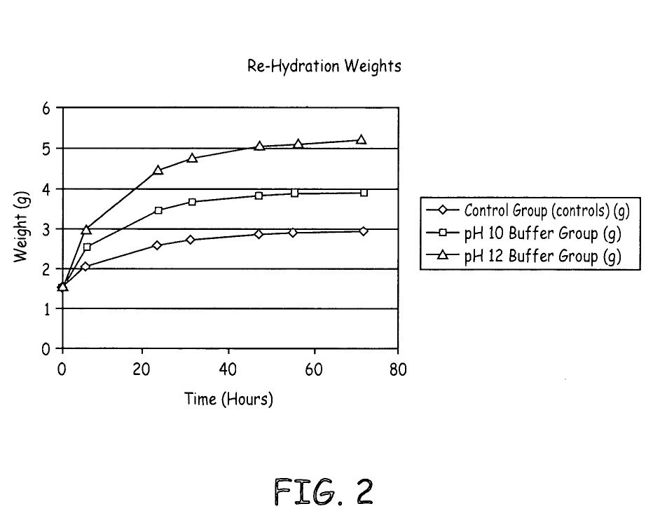

Samples were prepared using Hypan® hydrogel material in the form of small, elongated pellets (approximately 1.5 grams, dehydrated weight). Ten fully hydrated pellets were weighed and dimensions taken (height, width and length) before drying them in an oven at 79° C. for approximately 18 hours. After drying, the pellets were re-measured and divided into three groups. The first group of three pellets (“Control”) were not subjected to alkaline solution treatment, but instead were placed into deionized water. The second group of four pellets (“pH 10”) were placed in a pH 10 buffer solution. The third group of three pellets (“pH 12”) were placed in a pH 12 buffer solution. All submerged pellets were allowed to hydrate (at a temperature of approximately 37° C. until they reached full hydration (characterized by weight equilibrium). The pellets were then dehydrated in an oven at 79° C. for at least 18 hours. Subsequently, all pellets were placed in deionized water and allowed to re-hydrate. Weight and dimensional measurements were taken twice daily during the hydration period, and when weight values reached equilibrium (shown by paired t-tests), the pellets were determined to be at full hydration. The average weight measurements of the three sample groups are provided in Table 1, and are plotted in FIG. 2. The alkaline solution treated hydrogel pellets exhibited elevated swelling rates and elevated equilibrium swelling levels as compared to the Control group. For example, the pH 10 sample group attained the Control group equilibrium weight (approximately 2.96 grams) within 8-12 hours and the pH 12 sample group attained the Control group equilibrium weight in approximately 6.5 hours; this is in contrast to the Control group time of 71.5 hours to reach 2.96 grams.

TABLE 1

| Time | Control | pH 10 | pH 12 |
|---|---|---|---|
| (hours) | (grams) | (grams) | (grams) |
| 0 | 1.48 | 1.51 | 1.49 |
| 6.5 | 2.08 | 2.56 | 2.93 |
| 23.5 | 2.64 | 3.51 | 4.51 |
| 31.5 | 2.74 | 3.68 | 4.79 |
| 47.3 | 2.88 | 3.86 | 5.08 |
| 55.5 | 2.91 | 3.90 | 5.14 |
| 71.5 | 2.96 | 3.95 | 5.24 |

### Example 2

Prosthetic Disc Nucleus with Alkaline Treated Hydrogel Core

Example prosthetic spinal disc nucleus devices were prepared pursuant to a design currently utilized by Raymedica, Inc. of Bloomington, Minn. in which a hydrogel core is encompassed by a constraining jacket (similar to FIG. 1). In particular, hydrogel cores were prepared using Hypan®. A first group (“Control”) of five prosthetic disc nuclei were prepared using these hydrogel cores without further additional treatment (i.e., not subjected to alkaline solution treatment; placed within a constraining jacket). A second group (“Group A”) of five prosthetic disc nuclei were prepared by first dehydrating the hydrogel cores and then placing the hydrogel cores in an alkaline solution having a pH of 12. The dwell time in the alkaline solution was five days. Subsequently, each of the treated hydrogel cores were dehydrated and placed in a woven constraining jacket. A third group (“Group B”) of five prosthetic disc nuclei were prepared by first dehydrating the hydrogel cores. The dehydrated hydrogel cores were each placed in a woven constraining jacket. The combination hydrogel core/constraining jacket was then placed in an alkaline solution having a pH of 12. The prostheses remained in the alkaline solution until the hydrogel core was fully hydrated (i.e., reached equilibrium weight; approximately five days). The hydrogel cores were then dehydrated. A fourth group (“Group C”) of five prosthetic disc nuclei were prepared by in a manner virtually identical to Group B described above, except that the alkaline solution dwell time was limited to two days. Finally, a fifth group (“Group D”) of five prosthetic disc nuclei were prepared by first dehydrating the hydrogel cores. The dehydrated hydrogel cores were each placed in a woven constraining jacket. The resulting prostheses were hydrated in water and then dehydrated. Finally, the dehydrated prostheses were immersed in an alkaline solution having a pH of 12, and the hydrogel cores allowed to fully hydrate (reached equilibrium weight). Following alkaline solution treatment, the hydrogel cores were dehydrated.

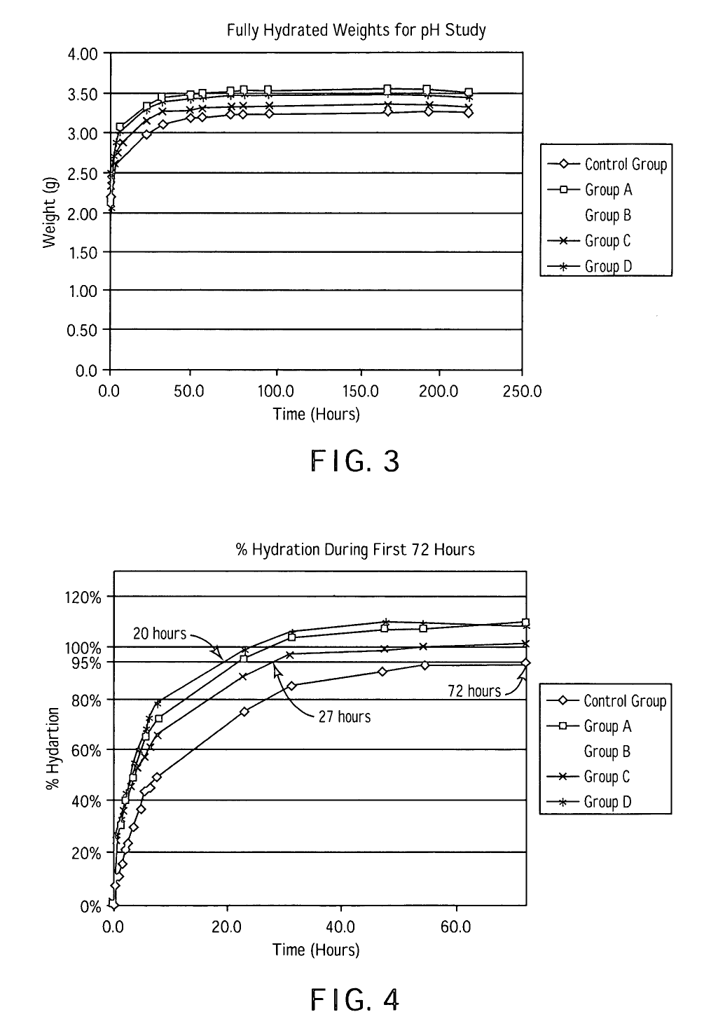

Each of the above prepared sample Groups were then placed in deionized water and allowed to re-hydrate. Weight and dimensional measurements were taken twice daily during the hydration period, and when weight values reached equilibrium (shown by paired t-tests), the pellets were assumed to be at full hydration. The average weight measurements of the five sample groups are provided in Table 2, and are plotted in FIG. 3.

TABLE 2

| | Control | Group A | Group B | Group C | Group D |
|---|---|---|---|---|---|
| Time (hours) | (grams) | (grams) | (grams) | (grams) | (grams) |
| 0.0 | 2.02 | 2.08 | 2.05 | 2.03 | 2.06 |
| 0.5 | 2.15 | 2.41 | 2.35 | 2.26 | 2.41 |
| 1.0 | 2.18 | 2.41 | 2.41 | 2.32 | 2.46 |
| 1.5 | 2.24 | 2.52 | 2.53 | 2.41 | 2.54 |
| 2.0 | 2.29 | 2.58 | 2.60 | 2.46 | 2.62 |
| 2.5 | 2.33 | 2.64 | 2.65 | 2.50 | 2.58 |
| 3.5 | 2.40 | 2.76 | 2.76 | 2.60 | 2.78 |
| 4.5 | 2.49 | 2.88 | 2.86 | 2.70 | 2.88 |
| 5.5 | 2.58 | 2.91 | 2.93 | 2.76 | 2.97 |
| 6.5 | 2.60 | 2.97 | 2.98 | 2.80 | 2.99 |
| 7.5 | 2.65 | 3.07 | 3.03 | 2.86 | 3.06 |
| 22.5 | 2.98 | 3.35 | 3.31 | 3.15 | 3.33 |
| 30.8 | 3.11 | 3.44 | 3.39 | 3.26 | 3.42 |
| 47.5 | 3.18 | 3.48 | 3.41 | 3.28 | 3.47 |
| 54.5 | 3.21 | 3.50 | 3.42 | 3.30 | 3.46 |
| 71.7 | 3.23 | 3.51 | 3.44 | 3.32 | 3.47 |
| 78.5 | 3.23 | 3.52 | 3.44 | 3.34 | 3.46 |
| 94.3 | 3.25 | 3.53 | 3.46 | 3.33 | 3.47 |
| 167.5 | 3.28 | 3.55 | 3.48 | 3.36 | 3.49 |
| 192.0 | 3.28 | 3.56 | 3.47 | 3.36 | 3.49 |
| 216.8 | 3.27 | 3.52 | 3.46 | 3.34 | 3.50 |

Of particular interest is the swelling rates or percent hydration over the first 72 hours, shown graphically in FIG. 4. As a point of reference, percent hydration was calculated as:

$$\frac{( current weight - initial weight ) / ( initial weight )}{\begin{matrix}( Control weight at 100 \% hydration - initial Control weight ) / \\ initial Control weight\end{matrix}}$$

After 72 hours, the Control group devices were at 95% hydration. Groups B and D appeared to reach 95% hydration after approximately 20 hours and then continued to hydrate to approximately 111% and 112%, hydration, respectively. Group A appeared to reach 95% hydration after 22 hours and continued to hydrate to approximately 114% hydration. Group C appeared to reach 95% hydration after 27 hours and then continued to hydrate to approximately 104% hydration. Thus, Group C exhibited an approximately 63% increase in swelling rate as compared to the Control Group; Group A exhibited an approximately 69% increase in swelling rate as compared to the Control Group; and Groups B and D exhibited an approximately 72% increase in swelling rate as compared to the Control Group.

### Example 3

Performance Analysis

Each of the samples prepared pursuant to Example 2 above were tested to determine whether a prosthetic disc nucleus incorporating an alkaline solution treated hydrogel core would perform properly under normal conditions experienced in an adult, human disc space. A common factor in proper device performance is the ability to absorb energy and maintain disc height following implant. In this regard, typical forces placed upon the disc space of an 180 pound adult range from 45 pounds (at rest) to 180 pounds (standing) to 360 pounds (lifting a heaving object). With this in mind, the samples of Example 2 were subjected to load-deflecting testing using an MTS compression tester. The load deflection test consisted of three cycles of loading to 500 pounds at a rate of 0.01 inches per second, with a 2-minute wait between each cycle. The energy absorbed by the devices at loads of 45 pounds, 180 pounds and 360 pounds were found by calculating the areas under the third load deflection curve for each sample and are provided in Table 3. In addition, the amount of compression of the device at each load was measured during the third loading cycle for each sample and are provided in Table 4. While small differences were noted, the functional performance of the treated devices was generally the same as the Controls.

TABLE 3

| | Energy @ | Energy @ | Energy @ |
|---|---|---|---|
| Group | 45 lb (N-m) | 180 lb (N-m) | 360 lb (N-m) |
| Control | 0.19 | 0.90 | 1.42 |
| A | 0.19 | 0.83 | 1.32 |
| B | 0.18 | 0.81 | 1.30 |
| C | 0.18 | 0.88 | 1.39 |
| D | 0.18 | 0.84 | 1.35 |

TABLE 4

| | Compression @ | Compression @ | Compression @ |
|---|---|---|---|
| Group | 45 lb (mm) | 180 lb (mm) | 360 lb (mm) |
| Control | 2.0 | 3.8 | 4.2 |
| A | 2.0 | 3.7 | 4.1 |
| B | 2.0 | 3.6 | 4.0 |
| C | 2.0 | 3.7 | 4.2 |
| DD | 2.0 | 3.6 | 4.1 |

The prosthetic spinal disc nucleus and method of manufacture thereof provides a marked improvement over previous designs. By treating the hydrogel core in an alkaline solution having a pH of at least about 8, most particularly about 10, the swelling rate and equilibrium swelling level are elevated, thereby minimizing the opportunity for prosthesis migration following implant.

Although the present invention has been described with reference to preferred embodiments, workers skilled in the art will recognize that changes can be made in form and detail without departing from the spirit and scope of the present invention.

What is claimed is:

1. A method of manufacturing a prosthetic spinal disc nucleus, the method comprising:
forming a hydrogel core from a hydrogel material having a natural swelling rate; and
treating the hydrogel core in a solution having a pH of greater than about 7 to transition the hydrogel core from a natural state to a treated state, wherein the hydrogel in the treated state exhibits an elevated swelling rate that is greater than the natural swelling rate.

2. The method of claim 1, further comprising:
inserting the hydrogel core into a constraining jacket.

3. The method of claim 2, wherein the hydrogel core is inserted into the constraining jacket before the step of treating the hydrogel core.

4. The method of claim 2, wherein the hydrogel core is inserted into the constraining jacket after the step of treating the hydrogel core.

5. The method of claim 1, wherein the step of treating the hydrogel core includes:
immersing a dehydrated or a hydrated hydrogel core in the solution; and dehydrating the hydrogel core.

6. The method of claim 5, wherein the alkaline solution has a pH of between about 8 and about 14.

7. The method of claim 1, wherein following the step of treating the hydrogel core, the elevated swelling rate is characterized by achieving 95% hydration in less than 50 hours, based upon an approximately 1.5 gram, dehydrated sample of the treated hydrogel core immersed in water.

8. The method of claim 7, wherein the natural swelling rate is characterized by a achieving 95% hydration after at least 72 hours, based upon an approximately 1.5 gram, dehydrated sample of the natural hydrogel core immersed in water.

9. The method of claim 1, wherein following the step of treating the hydrogel core, the elevated swelling rate is characterized by a reduction of at least 50% in time for a 1.5 gram, dehydrated sample to reach 95% hydration as compared to the natural swelling rate.

10. The method of claim 1, wherein the treated hydrogel core is characterized by releasing salt when subjected to an extraction process.

11. A method of manufacturing a prosthetic spinal disc nucleus, the method comprising:
forming a hydrogel core from a hydrogel material having a natural equilibrium swelling level; and
treating the hydrogel core in an alkaline solution having a pH of at least about 7.4 to transition the hydrogel core from a natural state to a treated state, where the hydrogel core in the treated state exhibits an elevated equilibrium swelling level that is greater than the natural equilibrium swelling level.

12. The method of claim 11, further comprising: inserting the hydrogel core into a constraining jacket.

13. The method of claim 12, wherein the hydrogel core is inserted into the constraining jacket before the step of treating the hydrogel core.

14. The method of claim 12, wherein the hydrogel core is inserted into the constraining jacket after the step of treating the hydrogel core.

15. The method of claim 11, wherein the step of treating the hydrogel includes:
immersing a dehydrated hydrogel or a hydrated hydrogel core in the alkaline solution; and dehydrating the hydrogel core.

16. The method of claim 11, wherein the alkaline solution has a pH of between about 8 and about 14.

17. The method of claim 11, wherein the elevated equilibrium swelling level is at least 110% for a device, 130% for the core alone of the natural equilibrium swelling level.

18. The method of claim 11, wherein the treated hydrogel core is characterized by releasing salt when subjected to an extraction process.

19. A method of manufacturing a prosthetic spinal disc nucleus, the method comprising:
forming a hydrogel core from a hydrogel material having a natural swelling rate and a natural equilibrium swelling level; and treating the hydrogel core in an alkaline solution having a pH of at least about 7.4 to transition the hydrogel core from a natural state to a treated state, wherein the hydrogel core in the treated state exhibits an elevated swelling rate that is greater than the natural swelling rate and an elevated equilibrium swelling level that is greater than the natural equilibrium swelling level.

20. An improved prosthetic spinal disc nucleus having a hydrogel core sized for implantation into a nucleus cavity and configured to hydrate from a dehydrated state to a hydrated state at natural swelling rate, the hydrogel core adapted to support opposing vertebrae in the hydrated state, the improvement comprising:
altering the hydrogel core to hydrate at an elevated swelling rate that is at least 125% greater than the natural swelling rate.

21. An improved prosthetic spinal disc nucleus having a hydrogel core sized for implantation into a nucleus cavity and configured to hydrated from a dehydrated state to a natural equilibrium swelling level adapted to support opposing vertebrae, the improvement comprising:
altering the hydrogel core such that the device hydrates to an elevated equilibrium swelling level that is at least 110% greater than the natural equilibrium swelling level.

22. A prosthetic spinal disc nucleus comprising a hydrogel core having cations incorporated into the hydrogel matrix, such that the swelling rate of the hydrogel core is increased relative to a hydrogel core devoid of such cations.

23. The prosthetic spinal disc nucleus of claim 22, wherein said cation is a metallic ion.

24. The prosthetic spinal disc nucleus of claim 22, wherein said cation is an organic ion.
